(19) United States
(12) Patent Application Publication
(10) Pub. No.: US 2026/0248629 A1
(43) Pub. Date: Aug. 27, 2026

(54) STENT GRAFT DELIVERY SYSTEMS, AND ASSOCIATED DEVICES AND METHODS

(71) Applicant: LimFlow S.A., Paris (FR)

(72) Inventors: Rowan Olund Hettel, Laguna Niguel, CA (US); Garrett Thomas Offerman, Orange, CA (US); Martin Clement, Chavornay (CH); Erik Wayne Delly, Paris (FR); John Michael Wainwright, Foothill Ranch, CA (US); Marcus Ian Tambongco Zozulenko, Rancho Mission Viejo, CA (US); Ravikiran Nataraj, Newport Beach, CA (US)

(21) Appl. No.: 19/441,651

(22) Filed: Jan. 6, 2026

Related U.S. Application Data
(60) Provisional application No. 63/761,837, filed on Feb. 21, 2025.
(60) Provisional application No. 63/788,638, filed on Apr. 14, 2025.

(51) Int. Cl. A61F 2/95; A61F 2/966

(52) U.S. Cl. CPC A61F 2/9517; A61F 2/966

(57) ABSTRACT

Disclosed herein are devices, systems, and methods for deploying an implantable device (e.g., a stent graft) within a patient. In some embodiments, a system in accordance with the present technology includes a catheter assembly and a handle. The catheter assembly can include an outer catheter, an intermediate catheter extending through the outer catheter, and an inner catheter extending through the intermediate catheter. In an undeployed position, a distal portion of the intermediate catheter can extend over and constrain the implantable device. The handle can include an actuator configured to retract the intermediate catheter to a deployed position in which the distal portion of the intermediate catheter does not constrain the implantable device. The handle can include a pulley assembly coupled between the actuator and the intermediate catheter to create a mechanical advantage that reduces the force required to actuate the actuator to retract the intermediate catheter.

### CROSS-REFERENCE TO RELATED APPLICATION

This application claims the benefit of (i) U.S. Provisional Patent Application No. 63/788,638, filed Apr. 14, 2025, and titled “STENT GRAFT DELIVERY SYSTEMS, AND ASSOCIATED DEVICES AND METHODS,” and (ii) U.S. Provisional Patent Application No. 63/761,837, filed Feb. 21, 2025, and titled “STENT GRAFT DELIVERY SYSTEMS,” which are each incorporated herein by reference in their entirety.

### BACKGROUND

Critical limb ischemia (CLI) is a severe form of peripheral artery disease (PAD) that can lead to significant morbidity. As CLI progresses to chronic limb-threatening ischemia (CLTI), patients may no longer be candidates for treatment via balloons or stents and may face bypass surgery, or worse, amputation.

Some minimally invasive procedures treat CLTI by endovascularly restoring blood flow to the affected limb by creating a new pathway for blood circulation by a process called percutaneous deep vein arterialization (pDVA). In such a procedure, a catheter is advanced into an artery of the leg including an arterial blockage, typically through a small incision in the groin. Another catheter is advanced into a vein in the leg, typically through the foot. The catheters are aligned with one another, and a needle is used to create an arteriovenous fistula between the artery and the vein. A guidewire is inserted through the needle, captured by the catheter in the vein, and retracted by the catheter in the vein to create a pathway from the artery to the vein for subsequent steps. In particular, yet another catheter including a stent graft can be advanced over the guidewire and used to deploy the stent graft in the arteriovenous fistula to maintain the patency thereof. Blood flows from the artery through the stent graft into the vein to perfuse the leg—bypassing the arterial blockage.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1 is a perspective view of a stent graft delivery system in accordance with embodiments of the present technology.

FIGS. 2A-2C are side views of a catheter assembly of the stent graft delivery system of FIG. 1 in an undeployed position, a partially-deployed position, and a deployed position, respectively, in accordance with embodiments of the present technology.

FIG. 3 is a cross-sectional view of an intermediate catheter of the catheter assembly of FIGS. 2A and 2B in accordance with embodiments of the present technology.

FIG. 4 is an inner side view of a handle of the stent graft delivery system in accordance with embodiments of the present technology.

FIGS. 5A and 5B are an inner side view and a perspective inner side view, respectively, of the handle of the stent graft delivery system in an undeployed position with a portion of the handle removed in accordance with embodiments of the present technology.

FIG. 6 is an enlarged perspective inner side view of the handle of the stent graft delivery system in accordance with embodiments of the present technology.

FIG. 7 is a side view of a connector of the stent graft delivery system in accordance with embodiments of the present technology.

FIG. 8 is a cross-sectional view of the handle of the stent graft delivery system in accordance with embodiments of the present technology.

FIGS. 9A and 9B are inner side views of the handle of the stent graft delivery system in a partially-deployed position and a deployed position, respectively, and with the same portion of the handle removed as shown in FIGS. 5A and 5B in accordance with embodiments of the present technology.

FIGS. 10A and 10B are cross-sectional inner side views of the handle of the stent graft delivery system in accordance with additional embodiments of the present technology.

FIG. 11A is a side view of a wheel and a spool of the handle of the stent graft delivery system in the deployed position in accordance with embodiments of the present technology.

FIG. 11B is a graph of a change in mechanical force of a pulley assembly of the handle of the stent graft delivery system during operation in accordance with embodiments of the present technology.

FIGS. 12A, 12C, and 12E are front views of various configurations of the wheel and the spool of the stent graft delivery system 100 in accordance with embodiments of the present technology.

FIGS. 12B, 12D, and 12F are enlarged front views of the spools of FIGS. 12A, 12C, and 12E, respectively, in accordance with embodiments of the present technology.

FIG. 13 is a perspective side view of the handle of the stent graft delivery system with the same portion of the handle removed as shown in FIGS. 5A and 5B in accordance with embodiments of the present technology.

FIG. 14 is an inner side view of the wheel and a button of the stent graft delivery system in accordance with embodiments of the present technology.

FIGS. 15A and 15B are perspective side views of the button of the stent graft delivery system in the locked position and unlocked position, respectively, in accordance with embodiments of the present technology.

FIG. 16A is an inner side view of the handle of the stent graft delivery system in accordance with embodiments of the present technology.

FIG. 16B is an outer side view of the handle of the stent graft delivery system in accordance with embodiments of the present technology.

### DETAILED DESCRIPTION

Aspects of the present technology are directed generally to systems, devices, and methods for delivering (e.g., disposing, placing) an implantable device, such as a stent graft, within the veins and/or arteries of a patient. A delivery system configured in accordance with the present technology can include a catheter assembly and a handle coupled to the catheter assembly. The catheter assembly can include an inner catheter, an intermediate catheter, and an outer catheter. A distal portion of the intermediate catheter and a distal portion of the outer catheter can define a receiving space for receiving and constraining the implantable device (e.g., stent graft) in an undeployed position. The handle can include a pulley assembly coupling a wheel operable by a user to a shuttle coupled to the intermediate catheter. As such, when the wheel is rotated, the intermediate catheter is retracted relative to the inner catheter and the stent graft such that the stent graft is deployed from the system. In some aspects of the present technology, this configuration allows stent grafts to be placed within the veins and/or arteries of a patient while reducing the risks of kinks in the catheter assembly and the risks of damaging the stent graft during insertion of the catheter assembly into the patient. Further, the pulley assembly can create a mechanical advantage for the user operating the wheel, reducing the amount of force that is needed to retract the intermediate catheter and deploy the stent graft. In some aspects of the present technology, this can make the system easier to use and also reduce the risk of the user being unable to retract the intermediate catheter due to frictional forces between the intermediate catheter and the stent graft.

In some embodiments, the stent graft delivery system includes a handle and a catheter assembly with a strain reliever between the handle and the outer catheter. In some aspects of the present technology, the strain reliever creates a transition zone between the catheter assembly and the handle that reduces strain (e.g., torsion, shearing force) on the catheter assembly. This can inhibit kinking in the catheter assembly and/or mechanical failure.

In some embodiments, the intermediate catheter extends past a distal end of the outer catheter and has a tapered portion positioned outside of the outer catheter. At the tapered portion, the diameter of the intermediate catheter increases to the diameter of the outer catheter. The increased diameter of the distal portion of the intermediate catheter after the tapered portion can define the receiving space.

In some embodiments, the intermediate catheter houses an inner catheter coupled to a distal tip. A portion of the distal tip can be housed inside of the intermediate catheter and a different portion of the distal tip can have a diameter equal to the diameter of the intermediate catheter and be housed outside of the intermediate catheter. In some aspects of the present technology, housing a portion of the distal tip in the intermediate catheter can create a uniform diameter of the catheter assembly along the length of the catheter assembly. This can inhibit kinks from forming in the catheter assembly and allow for easier insertion of the catheter assembly into the patient.

In some embodiments, the distal tip can include an opening fluidly coupled to a lumen of the inner catheter. In some aspects of the present technology, the opening allows a guidewire to be threaded through the system during the operation of the system, which can facilitate the alignment of additional systems with the distal tip.

In some embodiments, the inner catheter can include a traction pad on an outer surface of the inner catheter and a stent graft can be positioned around the traction pad. In some aspects of the present technology, the traction pad inhibits the stent graft from compressing and twisting during loading and deployment of the stent graft. This can maintain the integrity of the stent graft (e.g., inhibit the development of holes, wear, and fatigue points) and improve deployment accuracy (e.g., the accuracy of the location of the stent graft after deployment).

In some embodiments, an additional catheter with a tapered portion can be placed around the inner catheter to inhibit the stent graft from sliding along a length of the inner catheter. In some aspects of the present technology, the tapered portion of the additional catheter can make loading of the stent graft easier. For example, the additional catheter can be slid away from a loading area during loading of the stent graft and then returned to its intended position afterward, creating more room for loading of the stent graft.

In some embodiments, the intermediate catheter can include a plurality of grooves on an inner surface of the intermediate catheter configured to reduce the contacting surface area between the intermediate catheter and the stent graft. In some aspects of the present technology, reducing the contact between the intermediate catheter and the stent graft can reduce the mechanical force needed to retract the intermediate catheter.

In some embodiments, a handle with a pulley assembly including a spool, a pulley, a shuttle, and a wire coupling the spool and the shuttle. The shuttle can be slidably coupled to the inner catheter and include a cable mating portion spaced apart from the catheter assembly. A cable can couple the spool to the shuttle via the cable mating portion. In some aspects of the present technology, offsetting the cable mating portion inhibits interference between the inner catheter and the cable while the shuttle is moving.

In some embodiments, the shuttle can be slidably coupled to the inner catheter and retracted by the pulley assembly. The handle can further include a plurality of ribs configured to reduce deflection of the inner catheter during retraction of the shuttle. In some aspects of the present technology, reducing deflection of the inner catheter when the system is in operation can reduce the risk of damage to the inner catheter, the catheter assembly, and/or the pulley assembly.

In some embodiments, the handle can further include fins configured to support some or all of the weight of the shuttle within the handle. In some aspects of the present technology, the fins can reduce the deflection of the inner catheter due to the weight of the shuttle during the operation of the system which can reduce the risk of damage to the inner catheter, the catheter assembly, and/or the pulley assembly.

In some embodiments, the plurality of ribs and fins can define through holes for the shuttle to pass through when being retracted. In some aspects of the present technology, the through holes restrict movement of the shuttle, the inner catheter, and/or the cable within the handle, decreasing the risk of kinks in the inner catheter and/or deflection of the inner catheter. Further, the fins stabilize the shuttle within the handle. In other aspects of the present technology, the fins and plurality of ribs in combination reduce the chance of kinks in the catheter assembly and improve the accuracy of deployment of a stent graft housed within the catheter assembly. Reducing the risk of kinks can also limit unintentional damage to the stent graft due to frictional forces and/or bends in the catheter assembly.

In some embodiments, the pulley assembly can generate mechanical advantage for the user such that the force applied to the wheel is less than the corresponding force applied to the shuttle. In some aspects of the present technology, this configuration makes it easier for the user to spin the wheel and operate the device. Further, making the wheel easier to spin reduces strain on the user and can allow the user to maintain a steadier position of the handle.

In some embodiments, as the pulley assembly is operated, the cable spools around the spool, increasing the effective diameter of the spool. As such, the ratio of diameters of the spool and the pulley decreases, decreasing the mechanical advantage the user operating the pulley assembly has. In some aspects of the present technology, the decrease in mechanical advantage can be beneficial to the system as the force required to retract the intermediate catheter decreases as the shuttle moves from an undeployed position to a deployed position (e.g., as the cable spools, as the shuttle and intermediate catheter are retracted). In some aspects of the present technology, aligning the value of these forces can reduce strain on the system and reduce the risk of mechanical failure.

In some embodiments, the spool can define a circumferential recess where the cable spools during operation of the system. In some aspects of the present technology, varying the size and shape of the circumferential recess can change the mechanical advantage of the pulley assembly to adapt the system to various applications.

In some embodiments, the button can include a shuttle flange configured to support the weight of the shuttle in an undeployed position. In some aspects of the present technology, the shuttle flange can reduce strain on the catheter assembly in the undeployed position.

In some embodiments, the button can include flanges that pass through the window and lock the button in an unlocked position. In the unlocked position, the flanges extend outside of the handle through the window. In some aspects of the present technology, this configuration allows the user to press the flanges together and force the button back through the window to return the button to the locked position.

In some embodiments, the wheel can include a plurality of ratchet teeth and a leaf spring configured to inhibit one-directional movement of the wheel. More specifically, movement of the wheel in the direction corresponding to an undeployed position is inhibited. In some aspects of the present technology, inhibiting movement in the direction of the undeployed position stops the user from pushing the intermediate catheter back into the outer catheter which can reduce the risk of the intermediate catheter causing kinks and/or damage to the stent graft and/or system and reduces the risk of the intermediate catheter inadvertently pushing the stent graft out of the system prior to full deployment (e.g. at an unintended location within the patient).

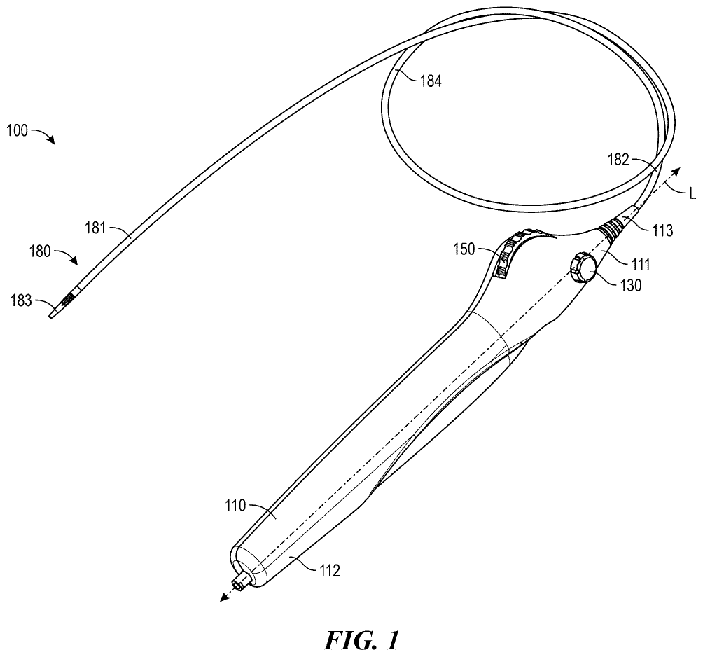

Specific details of several embodiments of the present technology are described herein with reference to FIGS. 1-16B. The present technology, however, can be practiced without some of these specific details. The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section. With regard to the terms “distal” and “proximal” within this description, unless otherwise specified, the terms can reference a relative position of the portions of the stent graft delivery system with reference to an operator and/or a location on the patient's skin. As used herein, the designations “rearward,” “forward,” “upward,” “downward,” “bottom,” “top,” “front,” “back,” and the like are not meant to limit the referenced component to a specific orientation. It will be appreciated that such designations refer to the orientation of the referenced component as illustrated in the Figures; the systems of the present technology can be used in any orientation suitable to the user.

Although reference is frequently made herein to delivery systems for deploying a stent graft, the present technology is further applicable to implanting and deploying other implantable devices. For example, the methods, systems, and devices described herein can be used to deliver and implant bare stents, prosthetic valves, and/or other medical devices.

The accompanying figures depict embodiments of the present technology and are not intended to be limiting of its scope. Depicted elements are not necessarily drawn to scale, and various elements can be arbitrarily enlarged to improve legibility. Component details can be abstracted in the figures to exclude details as such details are unnecessary for a complete understanding of how to make and use the present technology. Many of the details, dimensions, angles, and other features shown in the Figures are merely illustrative of particular embodiments of the disclosure. Accordingly, other embodiments can have other dimensions, angles, and features without departing from the spirit or scope of the present technology.

In the Figures, identical reference numbers identify identical, or at least generally similar, elements. To facilitate the discussion of any particular element, the most significant digit or digits of any reference number refers to the Figure in which that element is first introduced. For example, the handle 110 is first introduced and discussed with reference to FIG. 1.

The headings provided herein are for convenience only and should not be construed as limiting the subject matter disclosed. To the extent any materials incorporated herein by reference conflict with the present disclosure, the present disclosure controls.

### I. SELECTED EMBODIMENTS OF STENT GRAFT DELIVERY SYSTEMS

FIG. 1 is a perspective view of a stent graft delivery system 100 (“system 100”) in accordance with embodiments of the present technology. The system 100 can be used to endovascularly deliver and deploy a medical device, such as a stent graft, within a patient. In some embodiments, the system 100 can be used to deploy a stent graft to treat critical limb ischemia (CLI), critical limb-threatening ischemia (CLTI), and or other peripheral artery diseases according to any of the methods disclosed in, for example, (i) U.S. Pat. No. 10,398,580, filed Dec. 27, 2013, and titled “MINIMALLY INVASIVE SURGICAL APPARATUS AND METHODS”; (ii) U.S. Pat. No. 9,314,329, filed Aug. 25, 2015, and titled “METHODS AND SYSTEMS FOR PROVIDING OR MAINTAINING FLUID FLOW THROUGH BODY PASSAGES”; (iii) U.S. Pat. No. 9,545,263, filed May 21, 2015, and titled “DEVICES AND METHODS FOR TREATING LOWER EXTREMITY VASCULATURE”; (iv) U.S. Pat. No. 10,543,308, filed May 30, 2019, and titled “METHODS FOR ROUTING A GUIDEWIRE FROM A FIRST VESSEL AND THROUGH A SECOND VESSEL IN THE LOWER EXTREMITY VASCULATURE”; (v) U.S. Pat. No. 11,116,943, filed Apr. 8, 2021, and titled “METHODS FOR ACCESSING PEDAL VEINS”; and/or (vi) U.S. Pat. No. 11,612,397, filed Apr. 28, 2022, and titled “DEVICES AND METHODS FOR INCREASING BLOOD PERFUSION TO A DISTAL EXTREMITY”; each of which is incorporated by reference herein in its entirety.

In the illustrated embodiment, the stent graft delivery system 100 includes a handle 110 and a catheter assembly 180 coupled to the handle 110. The handle 110 is an elongated member with a distal portion 111 and a proximal portion 112. At the distal portion 111, the handle includes a strain reliever 113 coupled to at least a portion of the catheter assembly 180. The strain reliever 113 is a flexible member at the end of the handle 110 configured to flex with movement of the catheter assembly 180. In some aspects of the present technology, the strain reliever 113 creates a transition zone for the catheter assembly 180 that reduces strain (e.g., torsion, shearing force) on the catheter assembly 180. This transition zone can reduce the risk of mechanical failure of the catheter assembly 180 and the components housed within the catheter assembly 180. In some embodiments, the strain reliever 113 can be made of flexible materials such as rubber, polyethylene, and/or the like. In other embodiments, the strain reliever 113 can be omitted and the catheter assembly 180 can be reinforced where it is coupled to the handle 110. In still other embodiments, the strain reliever 113 can be made of hard plastic and can include a curved or chamfered distal end configured to reduce the strain on the catheter assembly 180.

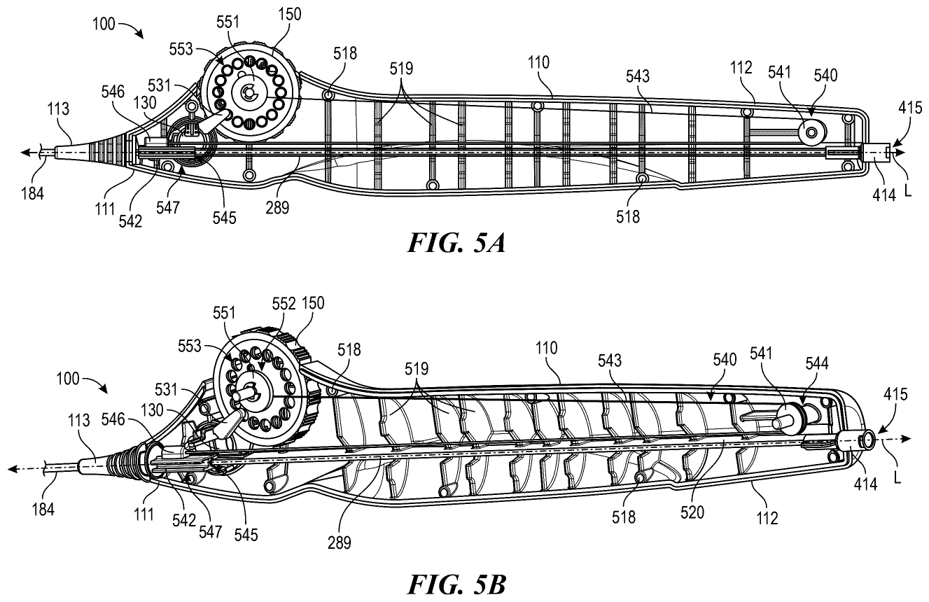

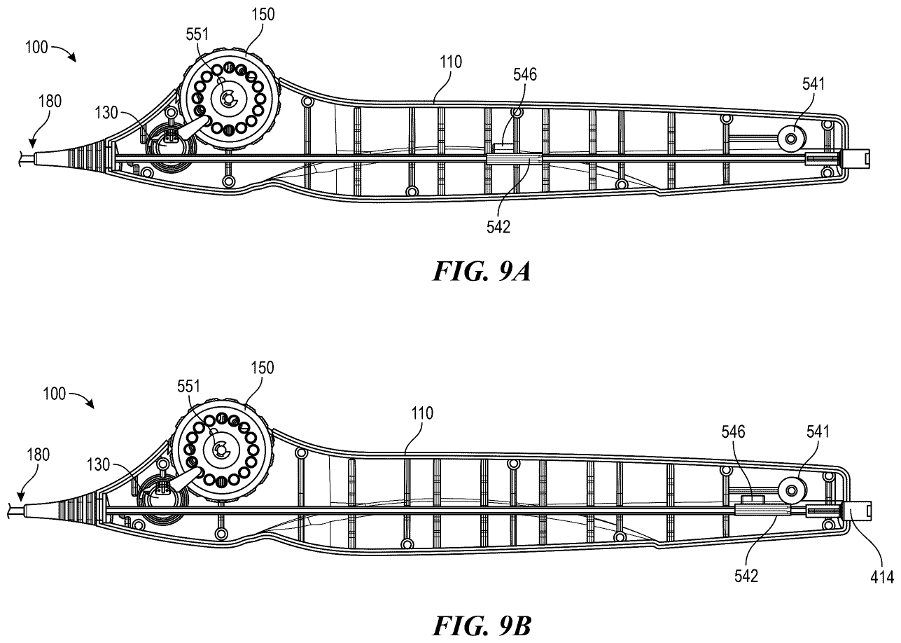

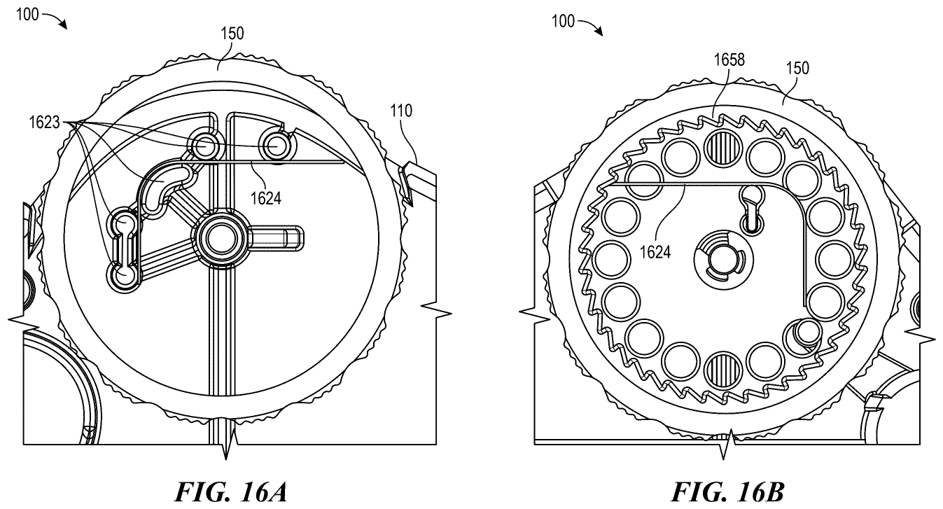

In the illustrated embodiment, the handle 110 further includes a button 130 (e.g., a lock, actuator, switch) and a wheel 150 (e.g., an actuator, a disk). The button 130 is operably coupled to the handle 110 and can move between a locked and unlocked position. In the illustrated embodiment, the button 130 is in a locked position. The button 130 is also operably coupled to the wheel 150 such that in the locked position, the wheel 150 cannot rotate and in the unlocked position, the wheel 150 can rotate, as described in greater detail below with reference to FIGS. 5A, 5B, and 13-15B. In other embodiments, the button 130 can be omitted. In some embodiments, the wheel 150 can include textures or features (e.g., grooves, bumps) that help a user grip an outer surface of the wheel 150 and rotate (e.g., spin) the wheel 150. For example, the wheel 150 can include features that enhance the user's grip in one direction and reduce the user's grip in the opposite direction to ensure the user rotates the wheel 150 in the desired direction. In the illustrated embodiment, the wheel 150 is rotationally coupled to the handle 110 such that the wheel 150 can rotate one-directionally around its center (e.g., counterclockwise), as described in greater detail below with reference to FIGS. 9A, 9B, 16A, and 16B. In other embodiments, the wheel 150 can freely rotate clockwise and counterclockwise. In some embodiments, the rotation of the wheel 150 is controlled by one-way rotational bearings coupled to the wheel 150. In other embodiments, the wheel 150 makes audible clicking noises as it is rotated to alert the user that the wheel 150 is changing positions.

In the illustrated embodiment, the handle 110 can be ergonomically shaped to facilitate the user comfortably wrapping their hand around the handle 110 between the distal portion 111 and the proximal portion 112. The shape of the handle 110 can also facilitate the user placing their thumb on the wheel 150 to operate the system 100, as described in greater detail below with reference to FIGS. 9A and 9B. In other embodiments, the user can operate the wheel 150 with other fingers. For example, in embodiments where the wheel 150 is placed on the bottom of the handle. In some embodiments, the handle 110 can be made of hard plastic and include rubber coverings to improve the user's ability to grip the handle 110 and/or user comfort.

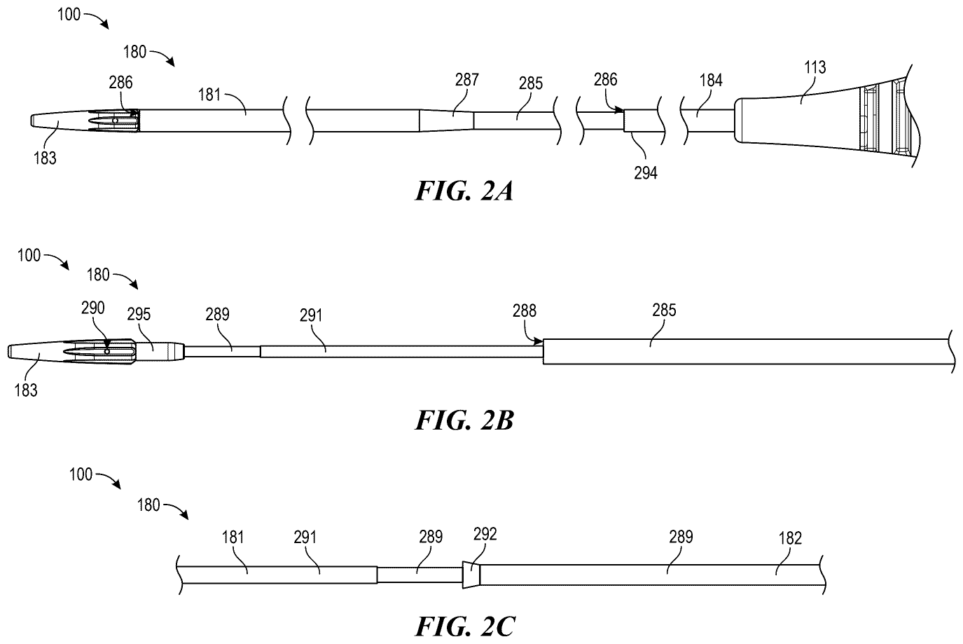

In the illustrated embodiment, the catheter assembly 180 includes a distal portion 181 and a proximal portion 182 adjacent to the distal portion 111 of the handle 110. The catheter assembly 180 is operably coupled to the handle 110, as described in greater detail below. At the distal portion 181, the catheter assembly 180 includes a distal tip 183. The distal tip 183 includes a rounded (e.g., curved, smooth) point (e.g., tip, end) configured to easily pass through the arterial and/or vascular system(s) of a patient without accidentally puncturing tissues. In some embodiments, the distal tip 183 can include features configured to retain a stent graft (e.g., implantable device) on (e.g., in, along) the distal portion 181 of the catheter assembly 180 before the system 100 is deployed. In the illustrated embodiment, the catheter assembly 180 includes an outer catheter 184 between the handle 110 and the distal tip 183 that houses additional catheters, as discussed in greater detail below with reference to FIGS. 2A-2C. The outer catheter 184 is fixedly coupled to the strain reliever 113 and does not extend inside the handle 110. In other embodiments, the outer catheter 184 can be coupled to other components and/or interior surfaces of the handle 110. In some embodiments, the outer catheter 184 can be thicker (e.g., wall thickness) and/or stiffer than other catheters of the catheter assembly 180 to reduce the chance of kinks developing in the catheter assembly 180. In further embodiments, the outer catheter 184 can have a constant diameter along a length of the outer catheter 184.

In some embodiments, the outer catheter 184 is a braided shaft (e.g., a braid-reinforced shaft) that extends about 80 centimeters from the handle 110. The catheter assembly 180 can have a size/diameter of about 10 French or less, about 8 French or less, about 7 French or less, about 5 French or less, or smaller. In some embodiments, the catheter assembly 180 has a size of 7 French or 6 French.

FIGS. 2A-2C are side views of the catheter assembly 180 of the stent graft delivery system 100 in an undeployed position, a partially-deployed position, and a fully-deployed position, respectively, in accordance with embodiments of the present technology. Referring to FIGS. 2A-2C together, the catheter assembly 180 comprises the outer catheter 184 (FIG. 1; e.g., an outer sheath, an outer elongate member, a first catheter, a first sheath, a first elongate member, and/or the like), an intermediate catheter 285 (FIGS. 2A and 2B; e.g., an intermediate sheath, an intermediate elongate member, a second catheter, a second sheath, a second elongate member, and/or the like) extending through the outer catheter 184, and an inner catheter 289 (FIGS. 2B and 2C; e.g., an inner sheath, an inner elongate member, a third catheter, a third sheath, a third elongate member, and/or the like) extending through the intermediate catheter 285. Referring also to FIG. 1, the intermediate catheter 285 is configured to be retracted via actuation of the wheel 150 to move the catheter assembly from the undeployed position (FIG. 2A) to the partially-deployed position (FIG. 2B) and from the partially-deployed position to the deployed position (FIG. 2C).

Referring to FIG. 2A, the intermediate catheter 285 extends through a lumen 286 of the outer catheter 184 and distally past a distal end 294 of the outer catheter 184. Accordingly, the outer catheter 184 does not extend the full length of the catheter assembly 180 and terminates proximally to the distal portion 181 of the catheter assembly 180. The intermediate catheter 285 can slide within the lumen 286 and can include a tapered portion 287 (e.g., flared portion) outside of the lumen 286 that can increase the diameter of the intermediate catheter 285 to be equal to or about equal to the diameter of the outer catheter 184. In some aspects of the present technology, the tapered portion 287 can reduce the risk of kinking in the catheter assembly 180 by keeping a diameter of the catheter assembly 180 generally uniform along a length of the catheter assembly 180. The increased diameter after the tapered portion 287 (e.g., at the distal portion 181) can also accommodate a stent graft (not shown) positioned inside of the intermediate catheter 285. The outer catheter 184 can have a lubricious inner surface to facilitate movement of the intermediate catheter 285 therethrough.

The tip 183 can engage (e.g., sealingly engage) a distal end of the intermediate catheter 285 at the distal portion 181 of the catheter assembly 180. In some embodiments, a proximal portion 295 (shown in FIG. 2B) of the distal tip 183 can be positioned within the intermediate catheter 285 in the undeployed position. More specifically, the proximal portion 295 of the distal tip 183 can extend into a lumen 288 of the intermediate catheter 285. In some embodiments, the proximal portion 295 of the distal tip 183 is omitted and the tip 183 can simply abut the distal end of the intermediate catheter 285. In some embodiments, features (e.g., protrusions) of the distal tip 183 can be configured to create friction between the distal tip 183 and the lumen 288 to help a portion of the distal tip 183 stay within the intermediate catheter 285 before the system 100 is deployed. This can inhibit kinking in the catheter assembly 180 and allow for easier insertion of the catheter assembly 180 into the patient.

Referring to FIG. 2B, in the partially-deployed position the intermediate catheter 285 is retracted proximally away from the tip 183 over the inner catheter 289. The inner catheter 289 extends within the lumen 288 of the intermediate catheter 285, and the intermediate catheter 285 can slide along an outer surface of the inner catheter 289. The inner catheter 289 can be fixedly coupled to the distal tip 183. In some embodiments, a lumen (not shown) of the inner catheter 289 can house a guidewire that can be threaded into the distal tip 183 and extend through an opening 290 (e.g., hole) of the distal tip 183. In some aspects of the present technology, the opening 290 allows a guidewire to be inserted through the catheter assembly 180 from the handle 110 during the operation of the system 100, which can facilitate alignment and positioning of the catheter assembly 180.

In the illustrated embodiment, a stent graft or other device (not shown) can be positioned between the intermediate catheter 285 and the inner catheter 289 for deployment by the system 100, as described in greater detail below with reference to FIGS. 9A and 9B. Such a stent graft can be of the type described in, for example, (i) U.S. Pat. No. 10,398,580, filed Dec. 27, 2013, and titled “MINIMALLY INVASIVE SURGICAL APPARATUS AND METHODS”; (ii) U.S. Pat. No. 9,314,329, filed Aug. 25, 2015, and titled “METHODS AND SYSTEMS FOR PROVIDING OR MAINTAINING FLUID FLOW THROUGH BODY PASSAGES”; (iii) U.S. Pat. No. 9,545,263, filed May 21, 2015, and titled “DEVICES AND METHODS FOR TREATING LOWER EXTREMITY VASCULATURE”; (iv) U.S. Pat. No. 10,543,308, filed May 30, 2019, and titled “METHODS FOR ROUTING A GUIDEWIRE FROM A FIRST VESSEL AND THROUGH A SECOND VESSEL IN THE LOWER EXTREMITY VASCULATURE”; (v) U.S. Pat. No. 11,116,943, filed Apr. 8, 2021, and titled “METHODS FOR ACCESSING PEDAL VEINS”; and/or (vi) U.S. Pat. No. 11,612,397, filed Apr. 28, 2022, and titled “DEVICES AND METHODS FOR INCREASING BLOOD PERFUSION TO A DISTAL EXTREMITY”; each of which is incorporated by reference herein in its entirety.

The stent graft can be in a compressed position when it is positioned inside of the intermediate catheter 285 (e.g., the undeployed position of FIG. 2A), in a partially-deployed position when its partially contained by the intermediate catheter 285 (e.g., the partially-deployed position of FIG. 2B), and in an expanded position when it is outside of the intermediate catheter 285 (e.g., the deployed position of FIG. 2C). More specifically, the stent graft can have a smaller diameter in the compressed position than in the expanded position. In the illustrated embodiment, a traction pad 291 is positioned on the outer surface of the inner catheter 289 between the intermediate catheter 285 and the inner catheter 289. The traction pad 291 can extend circumferentially around the inner catheter 289 or extend over only portions of a circumference of the inner catheter 289. The traction pad 291 can engage the stent graft during loading of the stent graft and deployment of the stent graft. The traction pad 291 can be “sticky” or otherwise configured to inhibit sliding of the stent graft. In some embodiments, the traction pad 291 can form an inner surface of the stent graft and can be deployed into the patient when the stent graft is deployed. In some aspects of the present technology, the traction pad 291 inhibits the stent graft from compressing and twisting during loading and deployment of the stent graft. This can maintain the integrity of the stent graft (e.g., inhibit the development of holes, wear, and fatigue points) and improve deployment accuracy (e.g., the accuracy of the location of the stent graft after deployment). In other embodiments, the traction pad 291 can be omitted and the stent graft can interface with the inner catheter 289 directly.

Referring to FIG. 2C, in the deployment position the intermediate catheter 285 (FIGS. 2A and 2B) is retracted proximally relative to the inner catheter 289 past a tapered portion (e.g., flared portion) or stop 292 of the inner catheter 289 to unsheathe and deploy the stent graft. The tapered portion 292 expands (e.g., increases in diameter) in the direction of the distal portion 181 of the catheter assembly 180. As such, when the stent graft is placed on the traction pad 291, the tapered portion 292 acts as a stop for the stent graft so that the stent graft is inhibited from moving relative to the inner catheter 289. More specifically, the tapered portion 292 inhibits the stent graft from moving towards the proximal portion 182 of the catheter assembly 180 and keeps the stent graft near the distal portion 181 as the intermediate catheter 285 (FIG. 2B) moves relative to the inner catheter 289, as described in greater detail below. Accordingly, a region of the inner catheter 289 from the tapered portion 292 to the tip 183 is configured to receive the stent graft thereover. The intermediate catheter 285 and the inner catheter 289 therefore form a capsule or receiving space along this region for receiving and securing the stent graft in the undeployed position.

Referring to FIG. 2C, the tapered portion 292 is integrally formed with the other portions of the inner catheter 289. For example, the tapered portion 292 can comprise part of an extrusion (e.g., a polyetheretherketone (PEEK) or other plastic extrusion) that extends over a hypotube or catheter structure. In other embodiments, the tapered portion 292 can be another catheter that surrounds the inner catheter 289 and is slidably coupled to the inner catheter 289. Referring to FIGS. 2A-2C, the inner catheter 289, the intermediate catheter 285, the outer catheter 184, the distal tip 183, and the stent graft are coaxially aligned.

Referring to FIG. 2A, the catheter assembly 180 is in the undeployed position. As such, the inner catheter 289, the traction pad 291, and the stent graft are fully housed/contained within the intermediate catheter 285. Referring to FIG. 2B, the catheter assembly 180 is in the partially-deployed position. As such, the intermediate catheter 285 has begun to retract through the outer catheter 184. As the intermediate catheter 285 retracts, the inner catheter 289, the traction pad 291, and the stent graft are exposed (e.g., unconstrained by the intermediate catheter 285). More specifically, the inner catheter 289, the traction pad 291, and the stent graft do not move relative to the intermediate catheter 285 as the intermediate catheter 285 is retracted. When the stent graft is exposed, it expands into the expanded position, separating slightly from the inner catheter 289 and/or the traction pad 291. Further, referring to FIGS. 2A and 2B, as the intermediate catheter 285 is retracted, the tapered portion 287 of the intermediate catheter 285 remains distal to the lumen 286 of the outer catheter 184. Referring to FIG. 2C, the catheter assembly 180 has been fully deployed, exposing the tapered portion 292 of the inner catheter 289. As such, the stent graft, which is distal the tapered portion 292, has been fully exposed, and its entire length has expanded into the expanded position. In the expanded position, the inner catheter 289, the distal tip 183, and the traction pad 291 can be pulled through a lumen of the stent graft, allowing the catheter assembly 180 to be removed from the patient while the stent graft remains in place (e.g., inside the body).

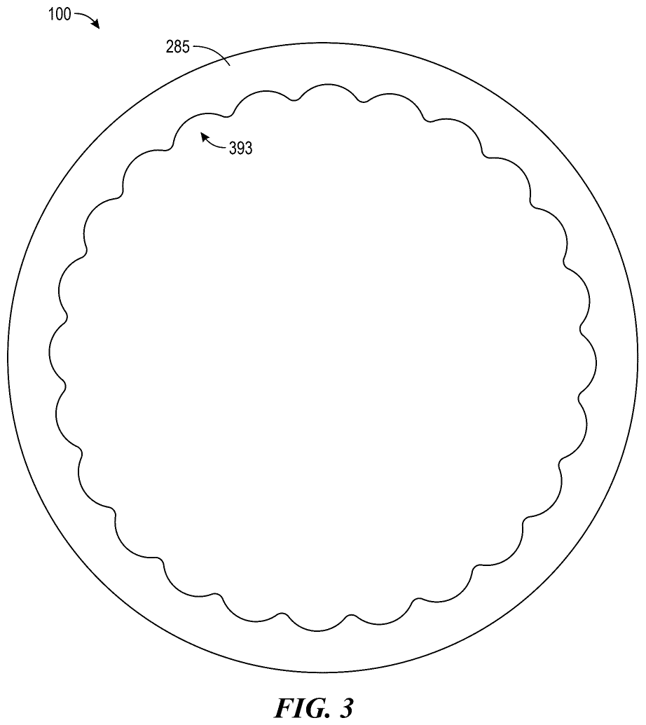

FIG. 3 is a cross-sectional view of the intermediate catheter 285 of the stent graft delivery system 100 in accordance with embodiments of the present technology. In the illustrated embodiment, the intermediate catheter 285 defines a plurality of semicircular grooves 393 positioned circumferentially around an inner surface of the intermediate catheter 285. The grooves can have a depth (e.g., distance from bottom to apex) of 0.001 inches, 0.0015 inches, 0.002 inches, or any depth therebetween. The plurality of grooves 393 can be configured to reduce surface-to-surface contact between the intermediate catheter 285 and the stent graft. More specifically, at a bottom of the individual grooves 393, the intermediate catheter 285 does not contact the stent graft, and at an apex between the individual grooves 393, the intermediate catheter 285 contacts the stent graft. In some aspects of the present technology, reducing the contact between the intermediate catheter 285 and the stent graft can reduce the mechanical force needed to retract the intermediate catheter 285, as described in greater detail below. In other embodiments, the plurality of grooves 393 can be omitted. In some embodiments, the plurality of grooves 393 can be zigzag-shaped, repeating ovular shapes, and/or the like.

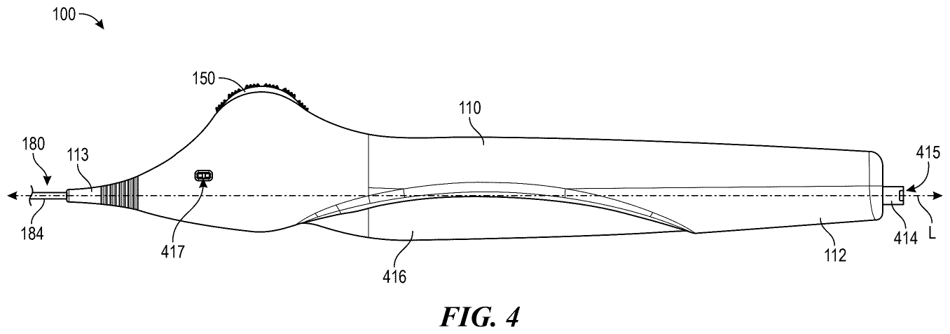

FIG. 4 is an inner side view of the handle 110 of the stent graft delivery system 100 in accordance with embodiments of the present technology. In the illustrated embodiment, the handle 110 includes a stopper 414 at the proximal portion 112. The stopper 414 defines a lumen 415 that can be coaxial with the strain reliever 113 along an axis L (e.g., a longitudinal axis L). The outer catheter 184 is fixedly coupled to the strain reliever 113, while the intermediate catheter 285 (FIGS. 2A and 2B) and the inner catheter 289 (FIGS. 2B and 2C) extend into the handle 110 as described in further detail below with reference to FIGS. 5A and 5B. In some embodiments, the axis L can be at the center of a cross-section of the handle 110, and in other embodiments, the axis L can be offset from the center of the handle 110. In the illustrated embodiment, the guidewire can be inserted through the lumen 415 and through the catheter assembly 180 to the opening 290 (FIG. 2B) of the distal tip 183 (FIG. 2B). The handle further includes finger grooves 416 configured to provide an ergonomic surface for the user to grab when operating the system 100. In some embodiments, the finger grooves 416 can be multiple recesses configured to fit individual fingers of the user's hand. In other embodiments, the finger grooves 416 can be positioned on different surfaces of the handle 110 to facilitate different configurations of the wheel 150. For example, the finger grooves 416 can be moved to the inner side of the handle (shown in FIG. 4) in embodiments where the user operates the wheel 150 with their forefinger.

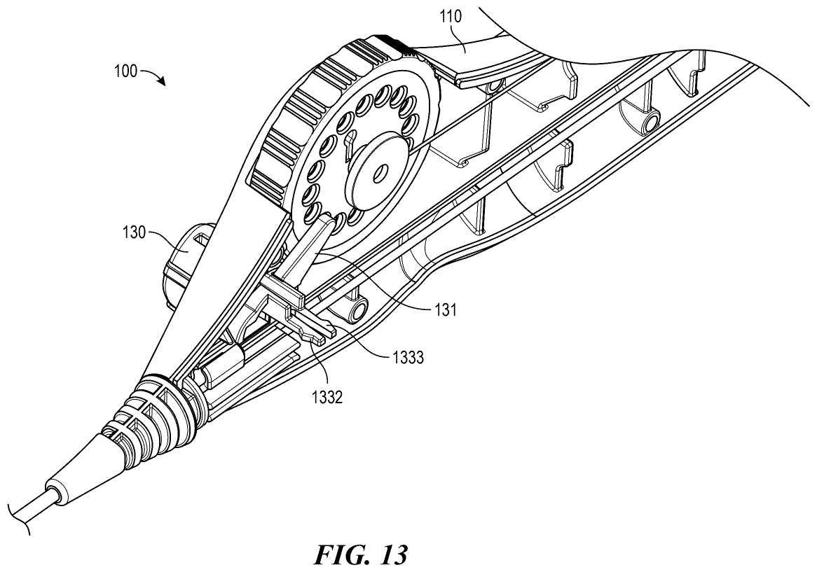

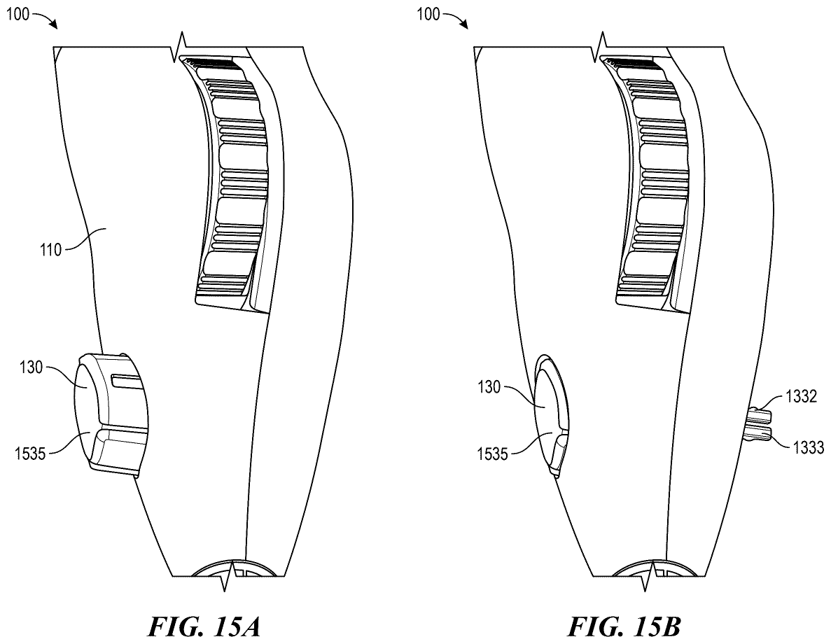

In the illustrated embodiment, the handle 110 further includes a window 417 (e.g., aperture, hole, opening). As described in further detail below with reference to FIGS. 13, 15A, and 15B, the window 417 is configured to allow features of the button 130 to pass through it when the button 130 (FIG. 1) is moved into the unlocked position. Further, the window 417 is configured to retain (e.g., restrain, inhibit movement of) those features until a user pinches (e.g., actuates, operates) them to release the button 130 from the unlocked position.

FIG. 5A is an inner side view of the handle 110 of the stent graft delivery system 100 in the undeployed position with a portion of the handle removed in accordance with embodiments of the present technology. FIG. 5B is a perspective inner side view of the handle 110 with the portion of the handle removed in the undeployed position in accordance with embodiments of the present technology. Referring to FIGS. 5A and 5B, the handle 110 includes a housing having a plurality of mating features 518 around a perimeter thereof. The plurality of mating features 518 can be configured to connect two opposing pieces of the handle 110 during manufacturing of the handle 110. For example, in the illustrated embodiment, the represented half of the handle 110 can be a first half, and the hidden half of the handle 110 can be a second half. The first half and the second half can mate together via the plurality of mating features 518. In other embodiments, the handle 110 can be a single piece or three or more pieces connected via a plurality of mating features 518. In some embodiments, the plurality of mating features 518 of the first half can be holes configured to receive extruding mating features of the second half. In other embodiments, the plurality of mating features 518 can be adhesives, snap-closed features, and/or the like.

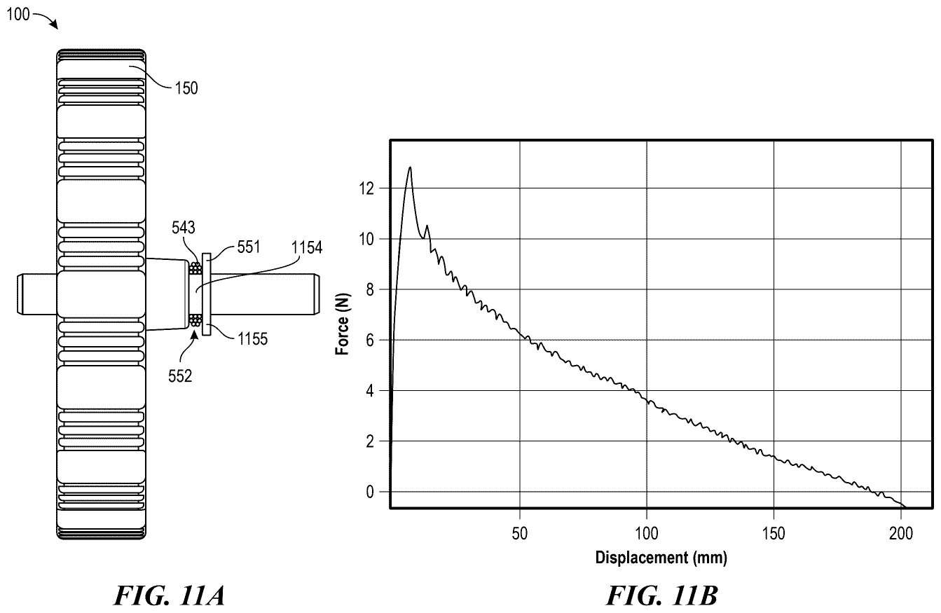

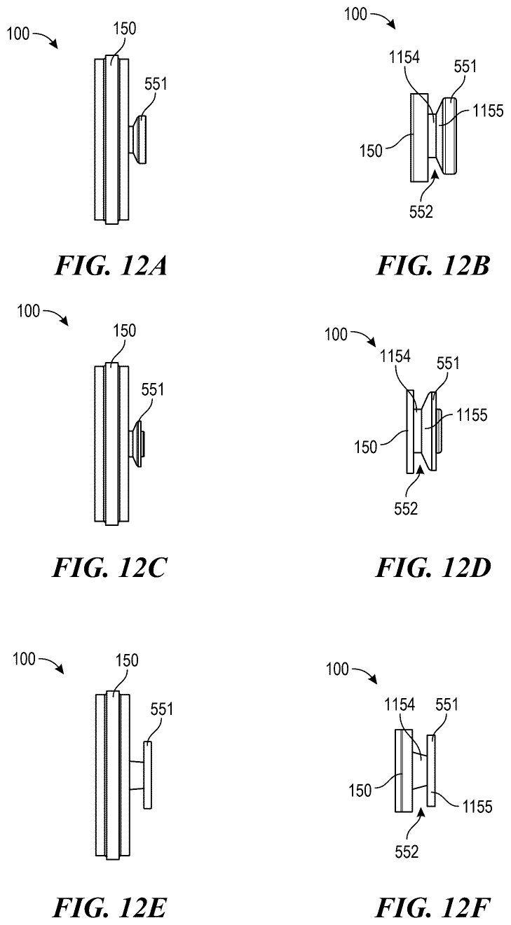

The handle 110 further includes a pulley assembly 540. The pulley assembly 540 includes a pulley 541 operably coupled to a spool 551 and a shuttle 542 (e.g., a connector) via a cable 543 (e.g., a wire, a string, a filament). In some embodiments, components of the pulley assembly 540 can be lubricated to reduce frictional forces within the pulley assembly 540. In the illustrated embodiment, the spool 551 is fixedly coupled to the wheel 150 and defines a circumferential recess 552 (FIG. 5B) configured to receive (e.g., hold, cradle) the cable 543, as described in greater detail below with reference to FIGS. 11A-12F. An end of the cable 543 can be fixedly coupled to the spool 551 mechanically or adhesively. In the illustrated embodiments, the spool 551 rotates at the same rotational speed as the wheel 150 and along the same axis as the wheel 150 (e.g., a central axis of the wheel 150). The pulley 541 is rotatably coupled to the handle 110 at the proximal portion 112 and defines a circumferential recess 544 (FIG. 5B) configured to receive (e.g., hold, cradle) the cable 543. The pulley 541 and cable 543 can have sufficient frictional force between the two to prevent the cable 543 from slipping (e.g., sliding) in the pulley 541. In some embodiments, the pulley 541 can include an opening in the center of the pulley that can be mated with an extruding member of the handle 110. Referring to FIGS. 5A and 5B, the pulley 541 rotates around a central axis of the pulley 541. More specifically, the wheel 150, the spool 551, and the pulley 541 rotate around axes perpendicular to the axis L.

In the undeployed position of FIGS. 5A and 5B, the shuttle 542 is located at the distal portion 111 of the handle 110. Referring to FIGS. 5A and 5B, the shuttle 542 defines a lumen 545 extending therethrough along the axis L. The inner catheter 289 extends through the lumen 545 to the stopper 414. As such, the shuttle 542 is slidably coupled to the inner catheter 289 and can slide along the length of the handle 110 along the axis L. The inner catheter 289 is fixedly coupled to the stopper 414 (e.g., a Leur connector) and the lumen of the inner catheter 289 aligns with the lumen 415 of the stopper 414 to allow the guidewire to pass through the handle 110, as described in detail above with reference to FIG. 4. Referring to FIGS. 5A and 5B, the shuttle 542 further includes a cable mating portion 546 configured to fixedly couple the shuttle 542 to the cable 543 at a location offset from the axis L. The cable 543 can be mechanically or adhesively coupled to the shuttle 542. In some aspects of the present technology, offsetting the cable mating portion 546 inhibits interference between the inner catheter 289 and the cable 543. A proximal end portion of the intermediate catheter 285 (FIGS. 2A and 2B) can be fixedly coupled to the shuttle 542. As such, when the shuttle 542 moves along the axis L from the undeployed position, the intermediate catheter 285 is drawn into the handle 110 and moves relative to the inner catheter 289 and the outer catheter 184.

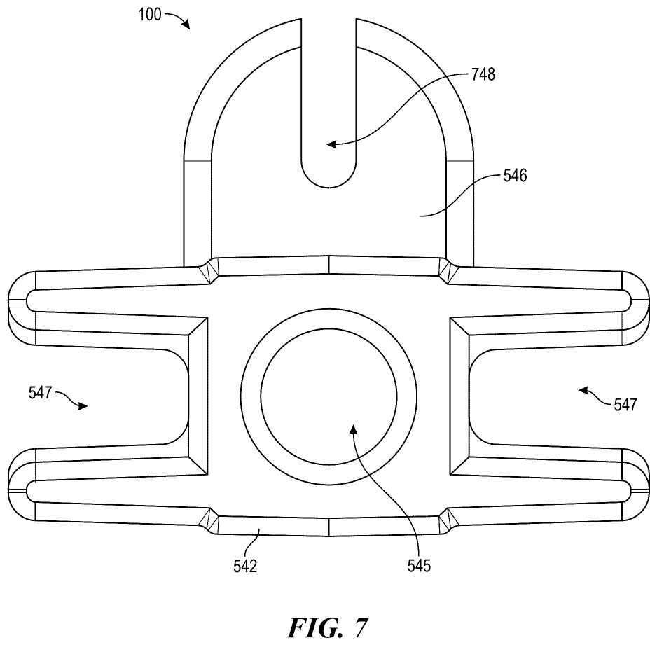

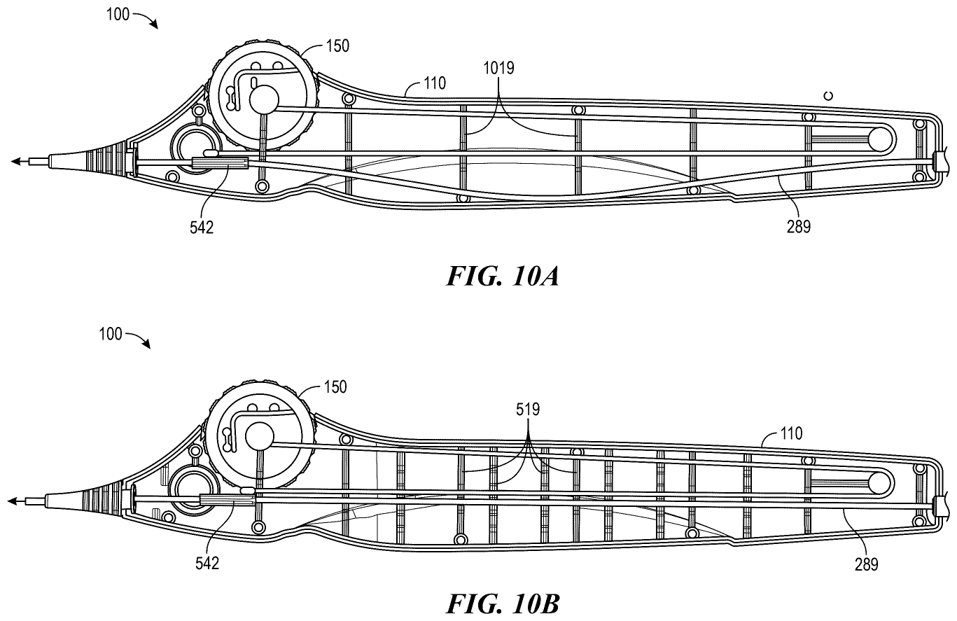

Referring to FIGS. 5A and 5B, the handle 110 (e.g., the housing thereof) further includes a plurality of ribs 519 (e.g., containment features). The plurality of ribs 519 can include features (e.g., protrusions, recesses) that align with features (e.g., protrusions, recesses) of the shuttle 542. For example, the plurality of ribs 519 can project radially inward towards the axis L. The plurality of ribs 519 can also act as restraints (e.g., barriers, guides) for the inner catheter 289, as described in greater detail below with reference to FIGS. 10A and 10B. In some aspects of the present technology, the plurality of ribs 519 inhibits deflection of the inner catheter 289 when the system 100 is in operation. Deflection of the inner catheter 289 could damage the inner catheter 289, the catheter assembly 180, and/or the pulley assembly 540, thus reducing deflection benefits the system 100. In the illustrated embodiment, the plurality of ribs 519 is a series of repeating individual ribs 519. In other embodiments, the gaps between the individual ribs 519 can be filled with additional material, such as foam; however, individual ribs 519 can reduce the weight of the handle 110 and make the operation of the system 100 less taxing on the user. In other embodiments, the handle 110 can include fewer ribs 519 of thicker widths (e.g., length along the axis L). Similarly, the handle 110 includes fins 520 (FIG. 5B; e.g., opposed fins) that extend along the length of the handle 110 from the distal portion 111 to the proximal portion 112. In the illustrated embodiment, one fin 520 is shown and a second hidden fin 520 mirrors the illustrated fin 520. The fins 520 can align with (e.g., slidably mate with, project into) side recesses 547 (e.g., opposed recesses, pair of recesses) of the shuttle 542 (best shown in FIG. 7). Referring to FIGS. 5A and 5B, the plurality of ribs 519 and the fins 520 are integrally formed with the handle 110. In other embodiments, the plurality of ribs 519 and the fins 520 can be insertable members and the handle 110 can include openings configured to receive the plurality of ribs 519 and the fins 520.

Referring to FIGS. 5A and 5B, the button 130 includes a locking member 531 configured to operably mate with one of a plurality of locking holes 553 of the wheel 150, as described in greater detail below with reference to FIGS. 13-15B. More specifically, in the locked position, the locking member 531 enters one of the plurality of locking holes 553 to inhibit or even prevent the wheel 150 from rotating. In the unlocked position, the locking member 531 is pushed out of one of the plurality of locking holes 553 and the wheel 150 is free to rotate. Furthermore, as the wheel 150 rotates, different holes of the plurality of holes 553 align with the locking member 531, allowing the wheel 150 to be locked at multiple positions. For example, the wheel 150 could be rotated a half turn, partially deploying the stent graft, as described in greater detail below with reference to FIGS. 9A and 9B, and then locked again if the stent graft is not properly positioned to allow for user adjustment of the system 100 to correct the improper placement without risking the stent graft deploying further. Referring to FIGS. 5A and 5B, the button 130 is in the unlocked position. As such, the locking member 531 is not engaged (e.g., planarly aligned) with one of the plurality of locking holes 553.

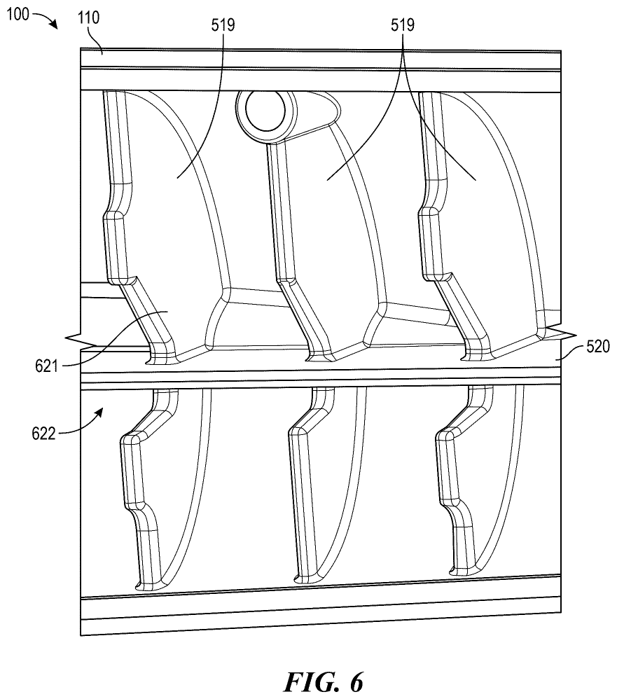

FIG. 6 is an enlarged perspective inner side view of the handle 110 of the stent graft delivery system 100 in accordance with embodiments of the present technology. In the illustrated embodiment, the plurality of ribs 519 includes containment portions 621 that define a plurality of through holes 622 that a portion of the cable 543 (FIG. 5A), the shuttle 542 (FIG. 5A), and the inner catheter 289 (FIG. 5A) can pass through. The containment portions 621 are generally the center portion of the individual ribs 519. The containment portions 621 can be angled to accommodate the cable mating portion 546 (FIG. 5A) of the shuttle 542. Further, the fin 520 extends into the through hole 622 to slidably mate with the side recesses 547 (FIG. 5A) of the shuttle 542. In other embodiments, the plurality of ribs 519 and the fin 520 can be shaped differently to accommodate different connector shapes. For example, the through holes 622 could be circular if the shuttle 542 is generally circular.

FIG. 7 is a side view of the shuttle 542 of the stent graft delivery system 100 in accordance with embodiments of the present technology. As described above, the shuttle 542 defines the lumen 545 and the side recesses 547 and includes the cable mating portion 546. In the illustrated embodiment, the cable mating portion 546 defines a wire recess 748 configured to receive the cable 543 (FIG. 5A) and fixedly couple the cable 543 to the shuttle 542. In some embodiments, the cable 543 can be tied around walls of the wire recess 748 or glued to the shuttle 542 in the wire recess 748. In some embodiments, the ends of the lumen 545 can be rounded or chamfered to inhibit damage or wear to the inner catheter 289 (FIG. 5A). In other embodiments, the side recesses 547 can be rectangular, circular, ovular, and/or the like and the fins 520 can be shaped accordingly. In the illustrated embodiment, as described above, the side recesses 547 are configured to slidably align with the fins 520 (FIG. 6). In some embodiments, the shuttle 542 and/or the fins 520 can be lubricated to reduce the force required to retract the shuttle 542.

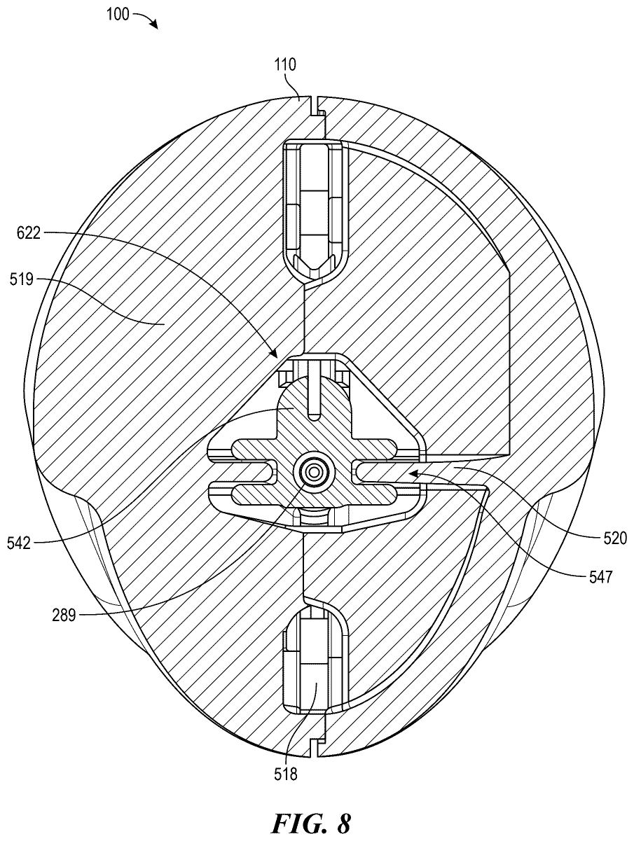

FIG. 8 is a cross-sectional side view of the handle 110 of the stent graft delivery system 100 in accordance with embodiments of the present technology. In the illustrated embodiment, the fins 520 extend into the side recesses 547 of the shuttle 542, and the shuttle 542 is positioned in the through holes 622 of the plurality of ribs 519 of the handle 110. The plurality of mating features 518 of the handle 110 are also mated. In some aspects of the present technology, the containment portions 621 inhibit deflection of the inner catheter 289, decreasing the risk of kinks in the inner catheter 289 or deflection of the inner catheter 289. For example, referring to FIGS. 2A-2C and 8, friction between the intermediate catheter 285 and the stent graft can cause the inner catheter 289 to compress when the catheter assembly 180 is moved from the undeployed position toward the deployed position. That compression could cause deflection of the inner catheter 289 within the handle 110, but the containment portions 621 can provide a mechanical stop to such deflection. Further, the fins 520 stabilize the shuttle 542 within the handle 110.

FIGS. 9A and 9B are inner side views of the handle of the stent graft delivery system in the partially-deployed position and the deployed position, respectively, and with the same portion of the handle 110 removed as shown in FIGS. 5A and 5B in accordance with embodiments of the present technology. Referring to FIG. 9A, the shuttle 542 has been retracted (e.g., pulled back) halfway through the length of the handle 110 via the cable 543 (FIG. 5A). More specifically, the wheel 150 has been rotated, causing the cable 543 to collect on (e.g., spool about, twist around, wind about) the spool 551 of the wheel 150 and retract the shuttle 542 by the cable mating portion 546. The pulley assembly 540 creates a mechanical advantage such that the force required for the user to rotate the wheel 150 is reduced. Further, the cable 543 rotates around the circumferential recess 544 (FIG. 5B) of the pulley 541 to create a differential mechanical advantage, as described in greater detail below with reference to FIGS. 11A and 11B. Mechanical advantage generally means that less force is exerted on the wheel 150 by the user than is exerted on the shuttle 542 as a result. In some aspects of the present technology, this configuration makes it easier for the user to rotate the wheel 150 and operate the pulley assembly 540 and/or the system 100. Further, reducing the force required to rotate the wheel 150 reduces strain on the user and can allow the user to maintain a steadier position of the handle 110 relative to the catheter assembly 180. In some embodiments, the pulley assembly 540 can impart a mechanical advantage of 7:1 or greater.

Referring to FIG. 9B, the shuttle 542 has been retracted (e.g., pulled back) fully through the length of the handle 110 via the cable 543 (FIG. 5A) and rests against the stopper 414. More specifically, the wheel 150 has been rotated completely, causing the cable 543 to collect on (e.g., spool, twist around) the spool 551 of the wheel 150 and retract the shuttle 542 by the cable mating portion 546. Referring to FIGS. 2A-2C, 5A, 9A, and 9B, the system 100 can include at least three positions: the undeployed position, the partially-deployed position, and the deployed position. FIGS. 2A and 5A correspond to the undeployed position, FIGS. 2B and 9A correspond to the partially-deployed position, and FIGS. 2C and 9B correspond to the deployed position.

During the operation of the system 100, the system 100 moves from the undeployed position to the partially-deployed position and the deployed position. For example, referring to FIGS. 2A and 5A, the system 100 can begin in a locked position (e.g., the button 130 in an uncompressed position) and the catheter assembly 180 can be positioned within a patient while the handle 110 remains external to the patient. Referring to FIGS. 2A and 5A, once the catheter assembly 180 has been appropriately positioned inside the patient, the button 130 can be actuated (e.g., pressed, depressed) to permit the wheel 150 to rotate. Referring to FIGS. 2B and 9A, the user can then begin rotating the wheel 150 in a direction (e.g., clockwise) to twist the cable 543 around the spool 551 and pull the shuttle 542 and the intermediate catheter 285 backward via the pulley assembly 540 into the handle 110. As the intermediate catheter 285 is retracted, the inner catheter 289, the stent graft, and the traction pad 291 are exposed and the stent graft expands, as described in greater detail above with reference to FIGS. 2A-2C. Referring to FIGS. 2C and 9B, the user continues to rotate the wheel 150 until the shuttle 542 reaches the stopper 414 and the stopper 414 inhibits further movement of the shuttle 542 and the wheel 150. When the shuttle 542 reaches the stopper 414 in the deployed position, the tapered portion 292 of the inner catheter 289 is exposed (e.g., uncovered from inside of the intermediate catheter 285). As such, the stent graft is fully expanded. The user is alerted that the system 100 is in the deployed position when the wheel 150 can no longer be rotated. Once the system 100 is in the deployed position, the catheter assembly 180 can be removed from the patient and the stent graft will remain. More specifically, the expanded stent graft will be able to pass around the distal tip 183, the inner catheter 289, and/or the traction pad 291 without being retracted along with the catheter assembly 180.

FIGS. 10A and 10B are cross-sectional inner side views of the handle 110 of the stent graft delivery system 100 in accordance with additional embodiments of the present technology. Referring to FIG. 10A, the handle 110 includes a first plurality of ribs 1019. The first plurality of ribs 1019 do not include the containment portions 621 (FIG. 6) or the through holes 622 (FIG. 6). In effect, the first plurality of ribs 1019 do not restrict movement of the inner catheter 289 away from the axis L within the handle 110. Accordingly, as shown, the inner catheter 289 may deflect significantly during the operation of the handle 110. Deflection typically occurs due to the high frictional force between the intermediate catheter 285 and the inner catheter 289, the outer catheter 184, the stent graft, and/or the traction pad 291 (FIGS. 2A-2C) during retraction of the shuttle 542 and the intermediate catheter 285. In effect, the intermediate catheter 285 catches (e.g., snags, grips) the inner catheter 289, traction pad 291 (FIG. 2B), and/or stent graft and pulls the inner catheter 289 further into the handle 110. This can lead to kinks in the inner catheter 289 and issues with full and accurate deployment of the stent graft. In contrast, FIG. 10B illustrates the arrangement of the ribs 519 described in detail above, including the containment portions 621 (FIG. 6). As shown, the deflection of the inner catheter 289 due to frictional forces is reduced. In some aspects of the present technology, this reduces kinks in the catheter assembly 180 (FIGS. 2A-2C) and improves the accuracy of stent graft deployment. Further, it can limit unintentional damage to the stent graft due to frictional forces.

FIG. 11A is a side view of the wheel 150 and the spool 551 of the stent graft delivery system 100 in the deployed position in accordance with embodiments of the present technology. In the illustrated embodiment, the spool 551 includes a spooling portion 1154 (e.g., a winding portion) extending from the wheel 150 and a flange portion 1155 (e.g., a washer) extending from the spooling portion 1154 and having a diameter greater than the spooling portion 1154. The cable 543 is configured (e.g., shaped and sized) to spool/wind about the spooling portion 1154 while the flange portion 1155 is configured (e.g., shaped and sized) to laterally constrain the cable 543 about the spooling portion 1154. In the illustrated embodiment, the cable 543 has been wrapped around the spool 551 multiple times during the deployment of the system 100. As such, the cable 543 has collected within the circumferential recess 552 of the spool 551 and slowly developed layers within the circumferential recess 552.

FIG. 11B is a graph of the change in mechanical force of the pulley assembly 540 as the stent graft delivery system 100 is operated. Referring to FIGS. 5B and 11B, the x-axis represents the displacement of the shuttle 542 within the handle 110. More specifically, as the value of the x-axis (e.g., the displacement) increases, the shuttle 542 is positioned closer to the stopper 414. The y-axis represents the force applied to the shuttle 542 via the wheel 150. As illustrated in FIG. 11B, the force applied to the shuttle 542 is greatest when the shuttle 542 is adjacent to the strain reliever 113 and none of the cable 543 has been wrapped around the spool 551. As the cable 543 wraps around the spool 551 and the shuttle 542 is retracted, the force applied to the shuttle 542 decreases. For example, when the shuttle 542 is fully retracted into the deployed position, the force applied to the shuttle 542 is at its lowest. More specifically, as the cable 543 is wrapped around the spool 551, the effective diameter of the circumferential recess 552 increases and the ratio of diameters of the circumferential recess 552 of the spool 551 and the circumferential recess 544 of the pulley 541 decreases. As this ratio decreases, mechanical advantage decreases, and more force is required from the user to apply the same force to the shuttle 542.

In some aspects of the present technology, the decrease in mechanical advantage can be beneficial to the system 100 as the force required to retract the intermediate catheter 285 decreases as the shuttle 542 moves from the undeployed position to the deployed position. This occurs because as the shuttle 542 is retracted, the intermediate catheter 285 retracts into the handle 110. Consequently, less surface area of the stent graft, the inner catheter 289, and/or the traction pad 291 contact the interior surface of the intermediate catheter 285, producing less frictional resistance to movement of the intermediate catheter 285 and the shuttle 542. Thus, spooling of the cable 543 reduces the force applied to the shuttle 542 as it is retracted, which aligns with the reduction in required force for retraction. Likewise, the decrease in mechanical advantage over the course of operation correspondingly increases the distance the shuttle 542 and the intermediate catheter 285 travel per unit of rotation of the wheel 150. That is, the rate of retraction of the shuttle 542 and the intermediate catheter increases as the force required to rotate the wheel 150 increases. Thus, the handle 110 is configured to provide the highest mechanical advantage initially when frictional forces are highest and decrease that mechanical advantage as the frictional forces decrease to provide greater travel distance per unit of rotation (e.g., increasing retraction rate), thereby reducing the time/effort needed to fully retract the intermediate catheter 285 to the deployed position. In some aspects of the present technology, aligning the value of these forces can reduce strain on the system 100 and reduce the risk of mechanical failure or improper stent graft deployment. In some embodiments, the pulley assembly 540 (FIG. 5A) can impart a mechanical advantage of 7:1 or greater initially, and impart a mechanical advantage of about 3:1 toward the end of the movement of the shuttle 542 to the deployed position (FIG. 9B).

In some embodiments, the cable 543 can have a variable diameter between the spool 551 and the shuttle 542 to increase the change/variance in mechanical advantage. For example, the cable 543 can be relatively thicker in diameter nearer to the shuttle 542 such that the mechanical advantage provided by the pulley assembly 540 decreases more quickly as the shuttle 542 moves closer to the deployed position. In such embodiments, the cable 543 can taper in diameter or have one or more sections with discrete, different diameters.

FIGS. 12A, 12C, and 12E are front views of various configurations of the wheel 150 and the spool 551 of the stent graft delivery system 100 in accordance with embodiments of the present technology. FIGS. 12B, 12D, and 12F are enlarged front views of the spools 551 of FIGS. 12A, 12C, and 12E, respectively, in accordance with embodiments of the present technology. Referring to FIGS. 12B and 12D, the spooling portion 1154 is generally cylindrical while the flange portion 1155 is tapered (e.g., chamfered) in a direction away from the wheel 150. Accordingly, the flange portion 1155 can direct the cable 543 (FIG. 11A) to spool about the spooling portion 1154 in multiple layers as described above. Likewise, the relatively narrow circumferential recess 552 defined between the flange portion 1155 and the wheel 150 can cause the cable 543 to spool about the spooling portion 1154 in multiple layers. Referring to FIG. 12F, the spooling portion 1154 can instead be tapered (e.g., chamfered) in a direction away from the wheel 150 to facilitate spooling of the cable 543 over itself within the circumferential recess 552. In some aspects of the present technology, the flange portion 1155 and/or the spooling portion 1154 can control (e.g., direct) the location where the cable 543 wraps around the spool 551, increasing the consistency of the outputted force from the pulley assembly 540 (FIG. 5A). In some embodiments, the spooling portion 1154 can be concave, convex, or otherwise shaped to optimize spooling of the cable 543 over itself.

In some aspects of the present technology, varying the size and shape of the circumferential recess 552 can change how the cable 543 spools around the spool 551 which can change the mechanical advantage of the pulley assembly 540. For example, in embodiments where the cable 543 collects faster (e.g., creates layers faster, fills the circumferential recess 552 faster), the force exerted on the shuttle 542 (FIG. 5A) via the pulley assembly 540 will decrease faster (and the travel distance of the shuttle 542 per unit rotation will correspondingly increase). This can be implemented in systems 100 where the stent graft is shorter and generates less frictional force to impede the movement of the intermediate catheter 285 (FIG. 2B) or in systems 100 where the traction pad 291 (FIG. 2B) is not used. In other aspects of the present technology, varying the size and shape of the spool 551 and/or circumferential recess 552 allows the force generated by the wheel 150 to be more closely aligned with the force required to retract the shuttle 542.

FIG. 13 is a perspective side view of the handle 110 of the stent graft delivery system 100 with the same portion of the handle 110 removed as shown in FIGS. 5A and 5B in accordance with embodiments of the present technology. In the illustrated embodiment, the button 130 includes flanges 1332 with locking portions 1333 configured to lock the button 130 into the window 417 (FIG. 4). In the locked position, the flanges 1332 are housed inside the handle 110. In the unlocked position, the flanges 1332 and locking portions 1333 are forced through the window 417 and the locking portions 1333 extend outside of the handle 110. For example, referring to FIGS. 4 and 13, during operation of the button 130, the flanges 1332 are pushed through the window 417 and bend (e.g., flex) inward to allow the locking portions 1333 to pass through the window 417. Once the button 130 has been fully actuated and the locking portions 1333 have passed through the window 417, the flanges 1332 return to their original position (e.g., unbent), and the locking portions 1333 extend past the perimeter of the window 417. As such, the button 130 cannot be returned to the locked position unless the user squeezes the flanges 1332 together and pushes the button 130 back through the window 417. More specifically, the user must squeeze the flanges 1332 enough for the locking portions 1333 to flex inward sufficiently to have a footprint that fits within the perimeter of the window 417.

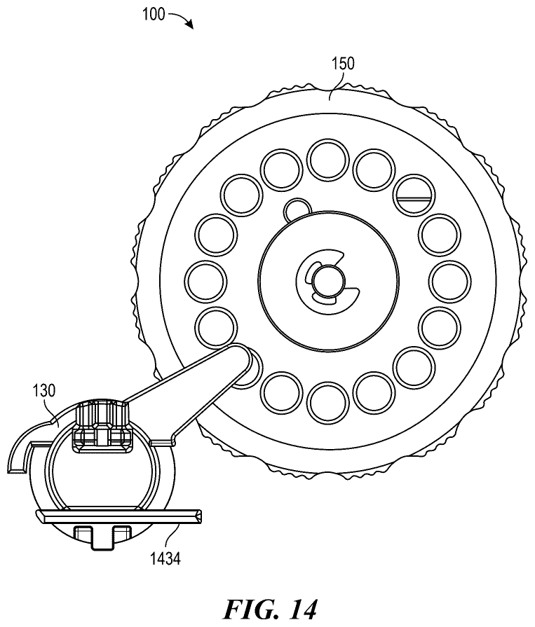

FIG. 14 is an inner side view of the wheel 150 and the button 130 of the stent graft delivery system 100 in accordance with embodiments of the present technology. In the illustrated embodiment, the button 130 includes a shuttle flange 1434 configured to be positioned within one of the side recesses 547 (FIG. 5A) of the shuttle 542 (FIG. 5A) when the button 130 is in the locked position. In some aspects of the present technology, the shuttle flange 1434 provides additional support to the shuttle 542 when the shuttle 542 is in the undeployed position, which can reduce strain on the catheter assembly 180 (FIG. 2A).

FIGS. 15A and 15B are perspective front side views of the button 130 of the stent graft delivery system 100 in the locked and unlocked position, respectively, in accordance with embodiments of the present technology. Referring to FIG. 15A, the button 130 includes an actuating surface 1535 where the user applies pressure to actuate (e.g., depress, push) the button 130 into the unlocked position. In the illustrated embodiment, the actuating surface 1535 is spaced apart from an exterior surface of the handle 110 for easy actuation. Further, the flanges 1332 are housed inside of the handle 110. Referring to FIG. 15B, the actuating surface 1535 is flush with the exterior surface of the handle 110 while the flanges 1332 and the locking portions 1333 extend outside of the window 417 (FIG. 4) of the handle 110. In some aspects of the present technology, this configuration allows the user to press the flanges 1332 together and force the flanges 1332 through the window 417 to return the button 130 to the locked position. Further, it allows the user to easily move the button 130 from the locked position to the unlocked position.

FIG. 16A is an inner side view of the handle 110 of the stent graft delivery system 100 in accordance with embodiments of the present technology. FIG. 16B is an outer side view of the handle 110 of the stent graft delivery system 100 in accordance with embodiments of the present technology. Referring to FIG. 16A, the handle 110 includes spring features 1623 (e.g., members, protrusions) configured to hold a leaf spring 1624 in a fixed position within the handle 110. Referring to FIG. 16B, the wheel 150 includes a plurality of ratchet teeth 1658. Referring to FIGS. 16A and 16B, the leaf spring 1624 is positioned so that a portion of the leaf spring 1624 engages with one of the plurality of ratchet teeth 1658 and inhibits the wheel 150 from rotating in one direction but permits rotation in the other direction. For example, referring to FIG. 16B, the plurality of ratchet teeth 1658 is configured to inhibit a user from rotating the wheel 150 clockwise. If a user were to try to rotate the wheel 150 clockwise, the leaf spring 1624 would be forced into one of the grooves between two of the plurality of ratchet teeth 1658, which would inhibit further rotation. In some aspects of the present technology, inhibiting clockwise movement of the wheel 150 stops the user from pushing the intermediate catheter 285 distally back through the outer catheter 185 (FIG. 2A) over the inner catheter 289 (FIGS. 2B and 2C; e.g., moving the system 100 from the deployed position to the undeployed position), which can reduce the risk of the intermediate catheter 285 causing kinks and/or damage to the stent graft and/or system 100 and reduces the risk of the intermediate catheter 285 inadvertently pushing the stent graft out of the system 100 prior to full deployment.

In other embodiments, the handle 110 includes features on an outer surface of the handle 110 configured to restrict the rotation of the wheel 150 to one direction. For example, the handle 110 can include ratchet teeth that are positioned adjacent to and in contact with an outer surface of the wheel 150. In some aspects of the present technology, external ratchet teeth could provide additional visual indicators to the user of the intended use of the system 100 (e.g., the intended rotational direction of the wheel 150) and/or louder audio indicators of movement of the wheel (e.g., clicking of the ratchet teeth).

### II. EXAMPLES

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.
1. A delivery system for deploying an implantable device, comprising:
a catheter assembly, wherein the catheter assembly comprises—
   an outer catheter having a distal end portion and a proximal end portion;
   an intermediate catheter extending through the outer catheter and having a distal end portion and a proximal end portion;
   an inner catheter extending through the outer catheter and having a distal end portion and a proximal end portion, wherein the distal end portion of the intermediate catheter and the distal end portion of the inner catheter (a) extend distally beyond the distal end portion of the outer catheter and (b) define a receiving space therebetween, and wherein the receiving space is configured to receive the implantable device therein; and
a handle, wherein the handle comprises—
   a housing, wherein the proximal end portion of the outer catheter and the proximal end portion of the inner catheter are fixedly coupled to the housing;
   a shuttle slidably positioned within the housing and fixedly coupled to the proximal end portion of the intermediate catheter;
   a rotatable actuator configured to be rotated by a user; and
   a pulley assembly including—
      a spool coupled to the rotatable actuator and configured to rotate with the rotatable actuator;
      a pulley rotatably coupled to the housing; and
      a cable extending from the spool, to and over the pulley, and from the pulley to the shuttle;
wherein rotation of the rotatable actuator is configured to wind the cable about the spool to pull the shuttle proximally through the housing to thereby retract the intermediate catheter proximally relative to the inner catheter and the outer catheter to release the implantable device from the receiving space.
2. The delivery system of example 1 wherein the inner catheter comprises a traction pad positioned on an outer surface of the inner catheter at the distal end portion of the inner catheter, and wherein the traction pad is configured to retain a position of the implantable device relative to the inner catheter when the intermediate catheter is proximally retracted.
3. The delivery system of either example 1 or example 2 wherein the spool is shaped and sized such that rotation of the rotatable actuator is configured to wind the cable about the spool in multiple layers to vary a mechanical advantage of the pulley assembly as the intermediate catheter is retracted proximally.
4. The delivery system of any of examples 1-3 wherein the housing includes a proximal portion and a distal portion, wherein the rotatable actuator is positioned proximate the distal portion of the housing, and wherein the pulley is positioned proximate the proximal portion of the housing.
5. The delivery system of any of examples 1-3 wherein the housing includes a proximal portion and a distal portion, wherein the proximal end portion of the outer catheter is fixedly coupled to the proximal portion of the housing, and wherein the proximal end portion of the inner catheter is fixedly coupled to the proximal portion of the housing.
6. The delivery system of any of examples 1-5 wherein the housing extends along a longitudinal axis, and wherein the housing includes a plurality of containment features projecting radially inward toward the longitudinal axis and configured to mechanically inhibit deflection of the inner catheter within the handle as the intermediate catheter is retracted proximally.
7. The delivery system of any of examples 1-6 wherein the shuttle has a pair of opposed recesses, and wherein the housing includes a pair of opposed fins configured to project into corresponding ones of the opposed recesses to slidably couple the shuttle to the housing.
8. The delivery system of any of examples 1-7 wherein the distal end portion of the intermediate catheter has a first diameter, and wherein the proximal end portion of the intermediate catheter has a second diameter smaller than the first diameter.
9. The delivery system of example 8 wherein the outer catheter has a constant diameter, and wherein the first diameter is equal to the constant diameter.
10. The delivery system of any of examples 1-9 wherein the rotatable actuator comprises ratchet teeth and a leaf spring configured to inhibit rotation of the rotatable actuator in one direction.
11. The delivery system of any of examples 1-10 wherein the implantable device is a stent graft.
12. The delivery system of any of examples 1-11 wherein the catheter assembly has a size of 6 French or less.
13. The delivery system of any of examples 1-12 wherein the inner catheter includes a flared portion configured to engage a proximal portion of the implantable device to inhibit proximal movement of the implantable device as the intermediate catheter is retracted proximally.
14. A delivery system for deploying an implantable device, comprising:
a catheter assembly, wherein the catheter assembly comprises—
   a first catheter; and
   a second catheter extending through the first catheter and having a distal end portion and a proximal end portion, wherein a distal portion of the first catheter and a distal portion of the second catheter define a receiving space therebetween, and wherein the receiving space is configured to receive the implantable device therein; and
a handle, wherein the handle comprises—
   a housing, wherein a proximal end portion of the second catheter is fixedly coupled to the housing;
   a shuttle slidably positioned within the housing and fixedly coupled to the proximal end portion of the first catheter;
   an actuator coupled to the housing; and
   a pulley assembly including—
      a spool coupled to the actuator, wherein actuation of the actuator is configured to rotate the spool;
      a pulley rotatably coupled to the housing; and
      a cable extending from the spool, to and over the pulley, and from the pulley to the shuttle;
wherein rotation of the actuator is configured to wind the cable about spool to pull the shuttle proximally through the housing to thereby retract the first catheter proximally relative to the second catheter from an undeployed position to a deployed position, wherein—
   in the undeployed position, the first catheter extends over the implantable device to constrain the implantable device in the receiving space, and
   during deployment, the first catheter is retracted proximally from the implantable device such that the implantable device is unconstrained by the first catheter.
15. The delivery system of example 14 wherein the spool defines a circumferential recess, and wherein rotation of the actuator is configured to wind the cable over itself within the circumferential recess.
16. The delivery system of example 15 wherein the circumferential recess includes an inner portion adjacent to the actuator and an outer portion, and wherein a diameter of the outer portion is greater than a diameter of the inner portion.
17. The delivery system of any of examples 14-16 wherein the spool is shaped and sized such that rotation of the actuator is configured to wind the cable about the spool in multiple layers to vary a mechanical advantage of the pulley assembly as the first catheter is retracted proximally from the undeployed position to the deployed position.
18. The delivery system any of examples 14-17 wherein the spool is shaped and sized such that rotation of the actuator is configured to wind the cable about the spool in multiple layers to decrease a mechanical advantage of the pulley assembly and increase a rate at which the first catheter is retracted as the first catheter is retracted proximally from the undeployed position to the deployed position.
19. A method of implanting an implantable device within a patient, the method comprising:
inserting a catheter assembly of a delivery system into the patient in an undeployed position, wherein the catheter assembly comprises—
   an outer catheter having a distal end portion;
   an intermediate catheter extending through the outer catheter and having a distal end portion and a proximal end portion; and
   an inner catheter extending through the outer catheter and having a distal end portion, wherein the distal end portion of the intermediate catheter and the distal end portion of the inner catheter (a) extend distally beyond the distal end portion of the outer catheter and (b) define a receiving space therebetween, wherein the receiving space is configured to receive and constrain the implantable device therein in the undeployed position;
actuating an actuator of a handle of the delivery system to retract the intermediate catheter relative to the inner catheter and the outer catheter to move the catheter assembly to a deployed position in which the implantable device is unconstrained by the intermediate catheter, wherein the intermediate catheter is coupled to a shuttle positioned within the handle, and wherein actuation of the actuator rotates a spool to pull a cable coupled to the shuttle over a pulley and wind the cable about the spool; and
removing the catheter assembly from the patient while the implantable device remains positioned in the patient.
20. The method of example 19 wherein the implantable device is a stent graft, and wherein the spool is shaped and sized such that actuation of the actuator is configured to wind the cable about the spool in multiple layers to vary a mechanical advantage created by the spool, the cable, and the pulley as the intermediate catheter is retracted proximally from the undeployed position to the deployed position.

### III. CONCLUSION

The above-detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

We claim:

1. A delivery system for deploying an implantable device, comprising:
a catheter assembly, wherein the catheter assembly comprises—
an outer catheter having a distal end portion and a proximal end portion;
an intermediate catheter extending through the outer catheter and having a distal end portion and a proximal end portion;
an inner catheter extending through the outer catheter and having a distal end portion and a proximal end portion, wherein the distal end portion of the intermediate catheter and the distal end portion of the inner catheter (a) extend distally beyond the distal end portion of the outer catheter and (b) define a receiving space therebetween, and wherein the receiving space is configured to receive the implantable device therein; and
a handle, wherein the handle comprises—
a housing, wherein the proximal end portion of the outer catheter and the proximal end portion of the inner catheter are fixedly coupled to the housing;
a shuttle slidably positioned within the housing and fixedly coupled to the proximal end portion of the intermediate catheter;
a rotatable actuator configured to be rotated by a user; and
a pulley assembly including—
a spool coupled to the rotatable actuator and configured to rotate with the rotatable actuator;
a pulley rotatably coupled to the housing; and
a cable extending from the spool, to and over the pulley, and from the pulley to the shuttle;
wherein rotation of the rotatable actuator is configured to wind the cable about the spool to pull the shuttle proximally through the housing to thereby retract the intermediate catheter proximally relative to the inner catheter and the outer catheter to release the implantable device from the receiving space.

2. The delivery system of claim 1 wherein the inner catheter comprises a traction pad positioned on an outer surface of the inner catheter at the distal end portion of the inner catheter, and wherein the traction pad is configured to retain a position of the implantable device relative to the inner catheter when the intermediate catheter is proximally retracted.

3. The delivery system of claim 1 wherein the spool is shaped and sized such that rotation of the rotatable actuator is configured to wind the cable about the spool in multiple layers to vary a mechanical advantage of the pulley assembly as the intermediate catheter is retracted proximally.

4. The delivery system of claim 1 wherein the housing includes a proximal portion and a distal portion, wherein the rotatable actuator is positioned proximate the distal portion of the housing, and wherein the pulley is positioned proximate the proximal portion of the housing.

5. The delivery system of claim 1 wherein the housing includes a proximal portion and a distal portion, wherein the proximal end portion of the outer catheter is fixedly coupled to the proximal portion of the housing, and wherein the proximal end portion of the inner catheter is fixedly coupled to the proximal portion of the housing.

6. The delivery system of claim 1 wherein the housing extends along a longitudinal axis, and wherein the housing includes a plurality of containment features projecting radially inward toward the longitudinal axis and configured to mechanically inhibit deflection of the inner catheter within the handle as the intermediate catheter is retracted proximally.

7. The delivery system of claim 1 wherein the shuttle has a pair of opposed recesses, and wherein the housing includes a pair of opposed fins configured to project into corresponding ones of the opposed recesses to slidably couple the shuttle to the housing.

8. The delivery system of claim 1 wherein the distal end portion of the intermediate catheter has a first diameter, and wherein the proximal end portion of the intermediate catheter has a second diameter smaller than the first diameter.

9. The delivery system of claim 8 wherein the outer catheter has a constant diameter, and wherein the first diameter is equal to the constant diameter.

10. The delivery system of claim 1 wherein the rotatable actuator comprises ratchet teeth and a leaf spring configured to inhibit rotation of the rotatable actuator in one direction.

11. The delivery system of claim 1 wherein the implantable device is a stent graft.

12. The delivery system of claim 1 wherein the catheter assembly has a size of 6 French or less.

13. The delivery system of claim 1 wherein the inner catheter includes a flared portion configured to engage a proximal portion of the implantable device to inhibit proximal movement of the implantable device as the intermediate catheter is retracted proximally.

14. A delivery system for deploying an implantable device, comprising:
a catheter assembly, wherein the catheter assembly comprises—
a first catheter; and
a second catheter extending through the first catheter and having a distal end portion and a proximal end portion, wherein a distal portion of the first catheter and a distal portion of the second catheter define a receiving space therebetween, and wherein the receiving space is configured to receive the implantable device therein; and
a handle, wherein the handle comprises—
a housing, wherein a proximal end portion of the second catheter is fixedly coupled to the housing;
a shuttle slidably positioned within the housing and fixedly coupled to the proximal end portion of the first catheter;
an actuator coupled to the housing; and
a pulley assembly including—
a spool coupled to the actuator, wherein actuation of the actuator is configured to rotate the spool;
a pulley rotatably coupled to the housing; and
a cable extending from the spool, to and over the pulley, and from the pulley to the shuttle;
wherein rotation of the actuator is configured to wind the cable about spool to pull the shuttle proximally through the housing to thereby retract the first catheter proximally relative to the second catheter from an undeployed position to a deployed position, wherein—
in the undeployed position, the first catheter extends over the implantable device to constrain the implantable device in the receiving space, and
during deployment, the first catheter is retracted proximally from the implantable device such that the implantable device is unconstrained by the first catheter.

15. The delivery system of claim 14 wherein the spool defines a circumferential recess, and wherein rotation of the actuator is configured to wind the cable over itself within the circumferential recess.

16. The delivery system of claim 15 wherein the circumferential recess includes an inner portion adjacent to the actuator and an outer portion, and wherein a diameter of the outer portion is greater than a diameter of the inner portion.

17. The delivery system of claim 14 wherein the spool is shaped and sized such that rotation of the actuator is configured to wind the cable about the spool in multiple layers to vary a mechanical advantage of the pulley assembly as the first catheter is retracted proximally from the undeployed position to the deployed position.

18. The delivery system of claim 14 wherein the spool is shaped and sized such that rotation of the actuator is configured to wind the cable about the spool in multiple layers to decrease a mechanical advantage of the pulley assembly and increase a rate at which the first catheter is retracted as the first catheter is retracted proximally from the undeployed position to the deployed position.

19. A method of implanting an implantable device within a patient, the method comprising:
inserting a catheter assembly of a delivery system into the patient in an undeployed position, wherein the catheter assembly comprises—
an outer catheter having a distal end portion;
an intermediate catheter extending through the outer catheter and having a distal end portion and a proximal end portion; and
an inner catheter extending through the outer catheter and having a distal end portion, wherein the distal end portion of the intermediate catheter and the distal end portion of the inner catheter (a) extend distally beyond the distal end portion of the outer catheter and (b) define a receiving space therebetween, wherein the receiving space is configured to receive and constrain the implantable device therein in the undeployed position;
actuating an actuator of a handle of the delivery system to retract the intermediate catheter relative to the inner catheter and the outer catheter to move the catheter assembly to a deployed position in which the implantable device is unconstrained by the intermediate catheter, wherein the intermediate catheter is coupled to a shuttle positioned within the handle, and wherein actuation of the actuator rotates a spool to pull a cable coupled to the shuttle over a pulley and wind the cable about the spool; and
removing the catheter assembly from the patient while the implantable device remains positioned in the patient.

20. The method of claim 19 wherein the implantable device is a stent graft, and wherein the spool is shaped and sized such that actuation of the actuator is configured to wind the cable about the spool in multiple layers to vary a mechanical advantage created by the spool, the cable, and the pulley as the intermediate catheter is retracted proximally from the undeployed position to the deployed position.
